(19) United States
(12) Patent Application Publication
(10) Pub. No.: US 2005/0282716 A1
(43) Pub. Date: Dec. 22, 2005

(54) Cleaning devices, systems, and methods for manufacturing the same, and products made by the system and method

(76) Inventor: Karen Papernik, Paterson, NY (US)

Correspondence Address: LACKENBACH SIEGEL, LLP, LACKENBACH SIEGEL BUILDING, 1 CHASE ROAD, SCARSDALE, NY, 10583, US

(21) Appl. No.: 11/156,835

(22) Filed: Jun. 20, 2005

Related U.S. Application Data
(60) Provisional application No. 60/580,891, filed on Jun. 18, 2004.

(51) Int. Cl. A61K 8/02

(52) U.S. Cl. 510/141

(57) ABSTRACT

The present invention provides a personal care and optionally a cleansing product for cleaning including a solid soap portion and a cleaning item molded partially into or secured within or in or two the solid soap portion, minimizing unintended separation during use. One or more cleaning items may be incorporated into a single solid soap portion increasing user convenience and suitability for a particular purpose (bath, emergency care, surgical, or selected work type). The soap product may be selected to include a plurality of additional non-soap items including scents, colors, moisturizers, grease and oil cutting agents, spices, anti-microbial agents etc.

### PRIOR APPLICATIONS

This application is related to and claims priority from U.S. Provisional Patent Application 60/580,891, filed Jun. 18, 2005, the entire contents of which are fully incorporated by reference.

### BACKGROUND OF THE INVENTION

1. Field of the Invention

The present invention relates to bath products. More particularly, the present invention relates to soap-based products that intermesh with other non-soap bath products and provide a unique convenience when combined.

2. Description of the Related Art

Many types of cleaning devices and products exist in the market. One subgroup of these cleaning products includes soaps, and other separate subgroups include scrubbers, brushes, physical exfoliatiors like facial scrub pads and woven mats, and foot treatment products like pumice stones.

In the previous art, exfoliatiors include sponges, pumice stone, and other abrasives, each provided alone to customers, or sold as sets with other bath items. These sets often include bath bags, buckets, sacks, and other containers for bathing equipment. Unfortunately, these containers while convenient for traveling and storage between bathing times, cannot prevent unintended separation during use.

During common use, a bather employs soap products individually to generate a lather when mixed with water and rubbed. Before or after initial soaping, users often abrade or clean their skin with separate brushes, sponges, pumice stones or other abrasives to smooth away rough skin and present a youthful appearance. Obviously this practice requires that the soap suds aid lubrication of the skin surface and transportation of loose skin away from the abrasion site.

It is common practice for bathers to misplace one or more of these bathing items during continued use. It is also common practice for bathers to require additional soap lubrication during abrasion and skin treatment. Such difficulties require that the step of abrasion cease during a re-application of soap to prevent skin damage due to over scrubbing or dry scrubbing.

As a result, the related art matter prior combinations with soap, which include holding supports namely rope, twine, and rigid wooden handles, are insufficient to respond to common bathing needs and increase the risk of skin damage.

### OBJECTS AND SUMMARY OF THE INVENTION

One object of the present invention is to provide a combined cleaning device incorporating a personal and physical cleaning product (e.g, brush, pumice stone, razor, trimmer, cleansing textile pads, brush, or other) with one or more soap products. The present invention may combine several cleaning devices or care products with a soap.

It is an additional object of the present invention, to provide a product easily adapted to medical, surgical, and special-use or special-need cleaning devices enabling simple sterile storage until use, or packaging for a particular purpose. For example, one of the cleaning items of the present invention (the soap/brush combo) may be stored in a sterile pack opened for washing up before a surgical operation, or used in emergency treatment of patients with road abrasions to both remove gravel and cleanse the skin. In an alternative embodiment, the above product may be combined with special oil-cutting soap and a brush for use in mechanical cleansing operations or fire damage recover operations.

The above, and other objects, features and advantages of the present invention will become apparent from the following description read in conduction with the accompanying drawings, in which like reference numerals designate the same elements.

### BRIEF DESCRIPTION OF THE DRAWINGS

FIG. 1 is a perspective view of one embodiment of the present invention containing a brush member and a soap portion.

FIG. 2 is a top view of the first embodiment shown in FIG. 1.

FIG. 3 is a sectional view along line I-I in FIG. 2.

FIG. 4 is perspective view of a second embodiment of the present invention containing a pumice stone member and a soap portion

FIG. 5 is a top view of FIG. 4.

FIG. 6 is a sectional view along line II-II in FIG. 5.

FIG. 7 is a perspective view of a third embodiment of the present invention containing an exfoliating member and a soap portion.

FIG. 8 is a top view of FIG. 7.

FIG. 9 is a sectional view along line III-III in FIG. 8.

### DESCRIPTION OF ONE PREFERRED EMBODIMENT

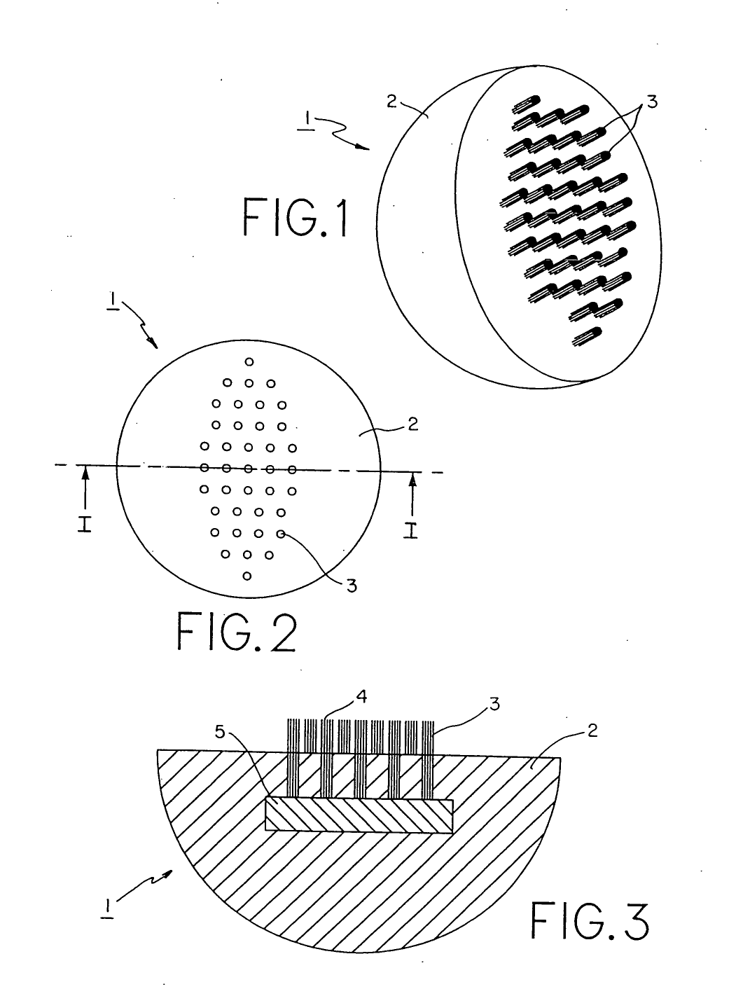

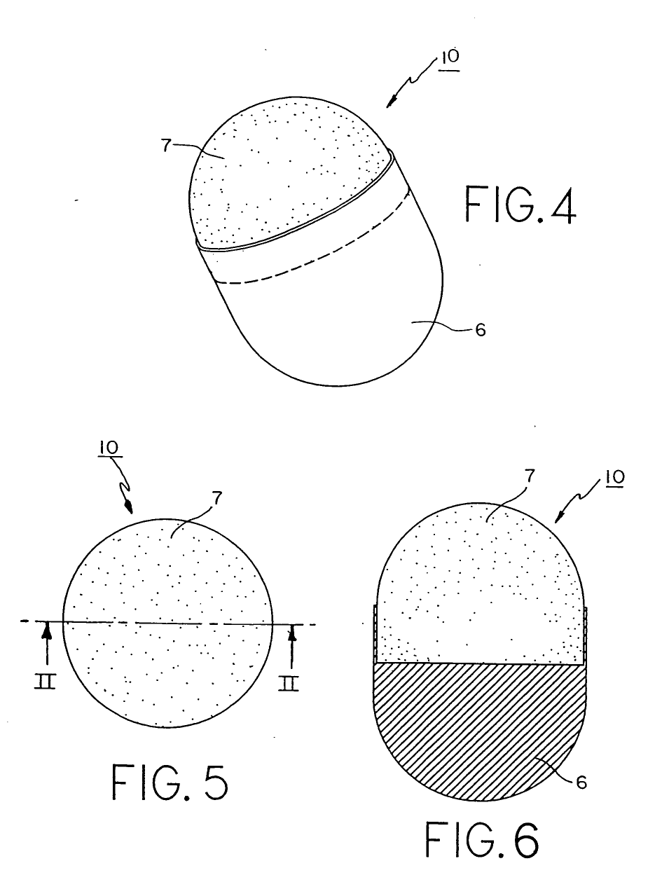

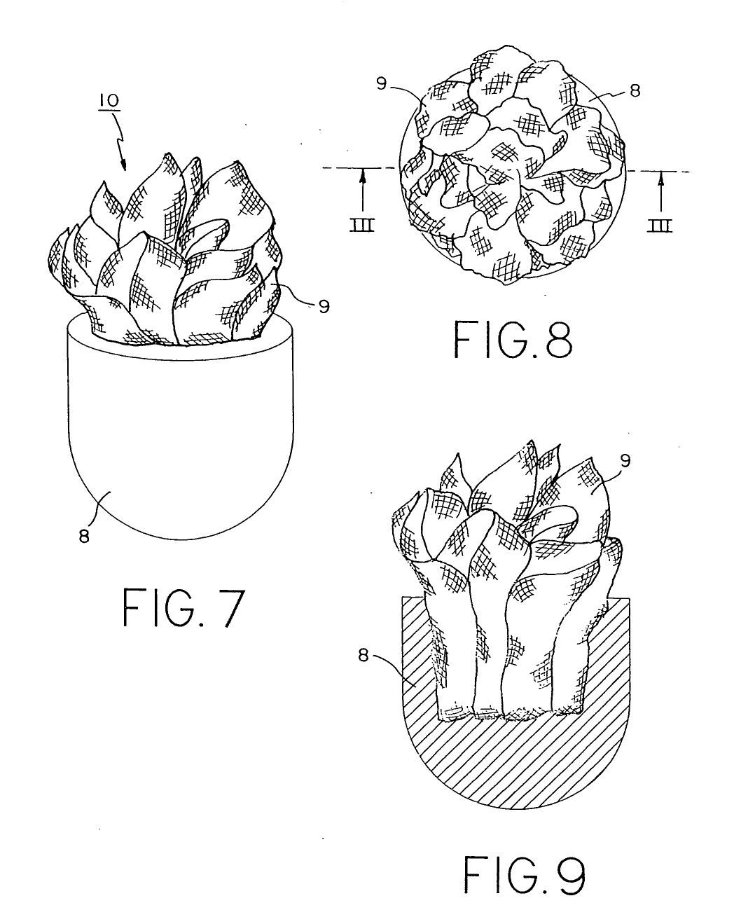

Referring generally to FIGS. 1 through 9, the present invention includes at least one personal or individual care member and a soap portion in a convenient hygiene tool, suitable for compact travel, and safe and specialized use in bathing, caring for patients, or addressing a selected cleaning task.

A cleaning device 1, includes a soap portion 2 and a brush member portion 3. Cleaning device 1 may be shaped in any convenient form for holding and using, for example the semi-circular shape shown, a round shape, a donut/torus shape, a rectilinear shape, or other reasonable shape.

Soap portion 2 may be constructed from any known soap product or assembly of products, including additional spices, scents, bubbles, plant parts, or other items. For example, soap portion 2 may include a particular scent or group of scents to appeal to a user's nose, have a special color or group of colors selected to complement brush member portion 3. Soap portion 2 may also include flower parts (rose petals, etc.), moisturizers, special scents, ropes or string for holding or hanging the product (not shown), an extending handle portion (not shown), or other parts known from the bath, surgical, and cleaning products trade. Soap portions 2 may be constructed in a “tears-free” formula or in a special hypoallergenic formula for sensitive skin.

In one alternative embodiment (not shown), a cleaning device 1 includes an extending wooden handle allowing a user to both soap and brush their back. It should be clear to those skilled in the soap making art that a plurality of items may be incorporated into the soap and the applicant intends to incorporate those items herein by reference into alternative embodiments.

Brush member portion 3 includes a plurality of bristle members 4 on one or more sides (as shown) and some form of bristle supporting/fixing/backing member 5. In some embodiments, supporting member 5 may include a handle or other common construction found in the art of cleaning and nail brushes. Here it is can be seen that soap portion 2 completely encloses supporting member 5 and portions of bristle members 4. In alternative embodiments (not shown), a portion of supporting member 5 may extend outward from soap portion 2, and may include an extending handle for scrubbing, a handle for easy grasping, or a hook for easy hanging or storage after use. The shape of supporting member 5 alternatively includes an engagement portion (not shown, such as an extending handle) enabling a more positive connection between the cast soap portion 2 and the brush member 3, minimizing unintended separation during use, particularly where the soap portion is reduced in size.

During construction, brush member portion 3 is placed in a mold containing liquid soap and allowed to harden and solidify. This embodiment, and all embodiments may be constructed in any common manner known to the soap molding and crating art.

During use, a user may grasp cleaning device 1 and soap a body portion, e.g., nails or hands, and then employ the brush member to scrub the nails or hands for additional cleaning power.

A second cleaning device 10 includes a soap portion 6 and a pumice stone member 7. In this embodiment, pumice stone member 7 is porous and is constructed to absorb portion of soap portion 6 to lock the two members together during use. A firm bonding is generated between pumice stone member 7 and soap portion 6 enabling long use periods without unintended separation. During use, a user softens a callus area with soap and water and then employs pumice stone member 7 to abrade the callus and create smooth skin.

As noted above the soap and cleaning member may be formed in any convenient shape and size and may include additional cleaning members (a separate brush or handle for example).

A third embodiment includes a third cleaning device 20 having a soap portion 8 and an exfoliating pad, poof, or woven member 9 constructed from plastic or other woven or formed material suitable for scrubbing and exfoliating the skin. This embodiment is formed by forcing a portion of woven member 9 into a liquid soap casting during the cooling process so that woven member 9 is firmly embedded withing soap portion 8.

During use, a user will soap an area to be scrubbed, and then employ woven member 9 to scrub and exfoliate the skin as desired.

It should be obvious to one skilled in the art that the present invention may be adapted to particular circumstances including bathing products, surgical preparation products, surgical patient preparation products, accident and emergency user products in transport packs, etc. The soap product may include special preservatives for long storage, anti-microbial ingredients, special colorants, and additional abrasives.

Alternative embodiments of the present invention involve the assembly of multiple cleaning devices in kits or assemblies in order to address a particular client need. For example, some kits may be constructed employing coordinated scents, or a specific course of skin treatments (exfoliating, cleaning, moisturizing, etc.), or a specific medical, industrial, or emergency need. It should be recognized that kits may also include other common cleaning items, including additional cleaning products or cleaning aids.

Although only a single or few exemplary embodiments of this invention have been described in detail above, those skilled in the art will readily appreciate that many modifications are possible in the exemplary embodiment(s) without materially departing from the novel teachings and advantages of this invention. Accordingly, all such modifications are intended to be included within the spirit and scope of this invention as defined in the following claims.

In any future claims, a means- or step-plus-function clause is intended to cover the structures described or suggested herein as performing the recited function and not only structural equivalents but also equivalent structures. Thus, for example, although a nail, a screw, and a bolt may not be structural equivalents in that a nail relies on friction between a wooden part and a cylindrical surface, a screw's helical surface positively engages the wooden part, and a bolt's head and nut compress opposite sides of a wooden part, in the environment of fastening wooden parts, a nail, a screw, and a bolt may be readily understood by those skilled in the art as equivalent structures. In a similar manner, any method type clauses requiring an action to result are not limited to any disclosed structures assistive to the action, but are to be understood to cover any action effective to carry out the method.

Having described at least one of the preferred embodiments of the present invention with reference to the accompanying drawings, it is to be understood that the invention is not limited to those precise embodiments, and that various changes, modifications, and adaptations maybe effected therein by one skilled in the art without departing from the scope or spirit of the invention as defined in the appended claims.

1. A personal care product, comprising
a soap member; and
at least a first abrasive personal care item integral bonded with said soap member, whereby during a use a user may employ are both said soap member and said first abrasive personal care item in a simultaneous manner allowing the generation of lubricating soap suds during a product use with water.

2. A personal care product, according to claim 1, wherein:
said first personal care item includes at least one of a brush, a pumice stone, an exfoliating member, and a woven member.

3. A personal care product, according to claim 2, wherein:
said woven member includes at least one of a cloth woven member, a plastic formed poof product, and an exfoliating grid.

4. A personal care product, according to claim 2, wherein:
said soap member is integrally bonded with at least a first and a second abrasive personal care item, wherein a combination of multiple personal care items allows an increased user convenient.

5. A method for manufacturing a cleaning product, comprising the steps of:
selecting a soap mold;
filling said soap mold with a liquid soap product;
selecting at least one exfoliating personal care item;
inserting a portion of said personal care item into said liquid soap product in said mold; and
allowing said soap product to solidify and removing said combined cleaning product from said mold.

6. A personal care kit, comprising:
a soap member; and
at least a first exfoliating personal care item integral bonded with said soap member, whereby during a use a user may employ are both said soap member and said first personal care item in a simultaneous manner allowing the generation of lubricating soap suds during a product use with water; and
said first personal care item includes at least one of a brush, a pumice stone, a rigid exfoliating member, and a woven member.

7. A personal care kit, according to claim 6, further comprising:
at least a second soap member;
said second soap member including at least a second exfoliating personal care item integrally bonded with said second soap member; whereby said personal care kit provides a plurality of soap members for user and manufacturer convenience.

8. A personal care kit, according to claim 7, further comprising:
at least a third soap member;
said third soap member including at least a third personal care item integrally bonded with said third soap member; whereby said personal care kit provides a plurality of soap members for user and manufacturer convenience.

9. A personal care kit, according to claim 8, further comprising:
a supporting member;
said supporting member providing a continuous support for each of said first, second, and third soap members combined with respective personal care items, whereby said supporting member enables a convenient packaging of said personal care kit and an improved marketability.
